(19) United States
(12) Patent Application Publication
(10) Pub. No.: US 2007/0173799 A1
(43) Pub. Date: Jul. 26, 2007

(54) Treatment of fatty tissue adjacent an eye

(76) Inventor: James C. Hsia, Weston, MA (US)

Correspondence Address: PROSKAUER ROSE LLP, ONE INTERNATIONAL PLACE 14TH FL, BOSTON, MA, 02110, US

(21) Appl. No.: 11/514,687

(22) Filed: Sep. 1, 2006

Related U.S. Application Data
(60) Provisional application No. 60/713,416, filed on Sep. 1, 2005.

(51) Int. Cl. A61B 18/04; A61B 18/18

(52) U.S. Cl. 606/27; 606/20

(57) ABSTRACT

The appearance of a sagging eyelid can be altered. A surface of a target region of skin of the eyelid is cooled, and a beam of radiation is delivered to the target region to affect at least one fat cell of the fatty deposit to alter the appearance of the sagging eyelid without causing substantial unwanted injury to tissue surrounding the target region of skin.

### CROSS-REFERENCE TO RELATED APPLICATION

This application claims priority to and the benefit of U.S. provisional patent application Ser. No. 60/713,416 filed Sep. 1, 2005, the entire disclosure of which is herein incorporated by reference.

### FIELD OF THE INVENTION

The invention relates generally to cosmetic treatments, and more particularly to using a beam of radiation to treat fatty tissue of an eyelid and/or adjacent an eye.

### BACKGROUND OF THE INVENTION

The eyes are surrounded by protective fat. The facial eyelid muscles and skin hold this retro-orbital fat in place resulting in a youthful line starting from the eyelashes to the cheek. Gravity and the wear of time can make all facial tissues sag, and the fat can begin to bulge. Some call this protrusion blepharochalasis, and it is more commonly called an eye bag or a baggy eyelid. An “under” eye bag can be accompanied by dark circles or discoloration, which can be caused by the appearance of blood in tissue surrounding the eye as this tissue thins as an individual ages or can be caused by shadows cast by bulging fat pockets. A fatty deposit can also form in an upper eyelid and cause it to sag.

Blepharoplasty (e.g., laser blepharoplasty) is a surgical procedure that can be used to treat eye bags. In a blepharoplasty procedure, a lower eyelid can be pulled away from the eyeball using a blunt retractor, while the eyeball is protected with a plastic plate. An electrocautery can be used to sweep across the conjunctiva (back side of the eyelid) along most of its length near its junction with the eyeball. The eyelid fat presents itself through the incision. The incision can be enlarged using scissors, if needed, and fat pockets can be individually teased out of their capsules and into the surgical field. The fat can then be clamped, excised, cauterized, and/or ablated by a laser in a conservative piecemeal fashion from each pocket to reduce the size or change the shape of the eye bag. Furthermore, because fat is being removed, extra baggy skin also can be excised to promote a more youthful appearance.

Due to the invasiveness of a blepharoplasty procedure, anesthesia is required to control pain, stitches can be used to facilitate healing, and post operative wound care can be required to avoid infection and scarring. In addition, post operative swelling can result, and significant recovery time can be required for a patient before returning to normal activity.

### SUMMARY OF THE INVENTION

The invention, in various embodiments, features a treatment for fatty tissue of an eyelid or fatty tissue proximate an eye. Instead of being an invasive surgical procedure, treatment radiation is directed through the surface of the skin. To alter the appearance of an eyelid, a treatment can, for example, reduce fat, remove a portion of fat, improve skin laxity, tighten skin, induce collagen formation, or perform some combination of the aforementioned. An advantage of a treatment that not only reduces or eliminates fatty tissue, but also tightens the skin, is that an invasive surgical procedure to excise the fat and extra baggy skin is not needed. Furthermore, using the invention, a treatment can include a series of treatment cycles, so that fatty tissue can be reduced gradually, and/or the skin can be tightened gradually, which further minimizes concerns about extra baggy skin under the eye. A treatment can include cooling to protect the skin surface, to minimize unwanted injury to the surface of the skin, and to minimize any pain that a patient may feel. An additional advantage of such a treatment is that the treatment can be performed with minimal cosmetic disturbance such that the patient can return to normal activity immediately after the treatment.

In one aspect, the invention features a method of altering the appearance of a sagging eyelid. The method includes cooling a surface of a target region of skin causing the sagging eyelid, and delivering a beam of radiation to the target region to affect at least one fat cell of a fatty deposit to alter the appearance of the sagging eyelid without causing substantial unwanted injury to tissue surrounding the target region.

In another aspect, the invention features an apparatus for altering the appearance of a sagging eyelid. The apparatus includes a source generating a beam of radiation. The source includes a fiber coupled laser diode array. A delivery system is coupled to the source for directing the beam of radiation to a target region of skin to affect at least one fat cell of a fatty deposit to alter the appearance of the sagging eyelid. A cooling system cools a surface of the target region of skin to minimize unwanted injury to tissue surrounding the target region.

In yet another aspect, the invention features an apparatus for altering the appearance of a sagging eyelid. The apparatus includes means for cooling a surface of a target region of skin causing the sagging eyelid, and means for delivering a beam of radiation to the target region to affect at least one fat cell of a fatty deposit to alter the appearance of the sagging eyelid without causing substantial unwanted injury to tissue surrounding the target region.

In other examples, any of the aspects above, or any apparatus or method described herein, can include one or more of the following features. In various embodiments, a fatty deposit of the eyelid can be reduced in size. A fat cell can be damaged so that lipid contained within can escape and at least a portion of the lipid can be carried away from the tissue. In some embodiments, the fat cell can be destroyed.

In various embodiments, the beam of radiation can be delivered to the target region to thermally injure the at least one fat cell. In certain embodiments, collagen formation can be induced in the target region of skin to improve skin laxity. Discoloration of skin of the target region can be improved or altered by a treatment.

In various embodiments, the beam of radiation is delivered to the target region about 0.1 mm to about 3 mm below the exposed surface of the skin. In certain embodiments, the beam of radiation can be focused below the surface of the skin in the target region to affect the at least one fat cell. A planoconvex lens can be used to focus the beam of radiation. A plurality of lens can be used to focus the beam of radiation.

In some embodiments, a lens with a concave contact surface is placed against the skin, and vacuum is applied to draw the target region of skin against the concave contact surface of the lens to focus the beam of radiation to the at least one fat cell in the target region.

The beam of radiation can have a wavelength of about 1,208 nm, 1,270 nm, 1,310 nm, 1,450 nm, 1,550 nm, 1,720 nm, 1750 nm, 1,930 nm, or 2,100 nm. In certain embodiments, a fiber coupled laser diode array generates the beam of radiation. The fiber coupled laser diode array can include a high power semiconductor laser.

The details of one or more examples are set forth in the accompanying drawings and the description below. Further features, aspects, and advantages of the invention will become apparent from the description, the drawings, and the claims.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows an exemplary system for treating tissue proximate an eye.

FIG. 2 depicts a planoconvex lens positioned on a skin surface.

FIG. 3 shows a plurality of lens focusing radiation to a target region of skin.

FIG. 4 shows a lens having a concave surface positioned on a skin surface.

### DESCRIPTION OF THE INVENTION

Radiation can be delivered to a target region of skin to target a fatty deposit. The target region of skin can include a portion of an eyelid (either a top eyelid or a bottom eyelid), tissue adjacent an eye, tissue surrounding an eye, tissue under or over an eye, tissue proximate an eye, or any combination of the aforementioned. In one embodiment, a treatment can alter or improve the appearance of a fatty deposit of the eyelid. For example, a treatment can alter the appearance of a sagging eyelid or reduce the size of a bag or a sack of an eyelid, e.g., an upper or lower eyelid.

In various embodiments, a treatment can reduce discoloration of skin of the target region. The beam of radiation treating the target region of skin can reduce the discoloration. In some embodiments, a second beam of radiation can be used to reduce the discoloration. For example, the second beam of radiation can target blood, blood vessels, and/or a component of blood to reduce the discoloration.

The fatty deposit can be disposed in a subcutaneous layer of fat or disposed in a dermal region of skin. In some embodiments, the beam of radiation is delivered through a surface of an epidermal region and penetrates through the epidermis and dermis to reach the subcutaneous fat. In certain embodiments, the beam of penetrates through the epidermis and into the dermis to treat fat in the dermal layer.

The treatment radiation can damage one or more fat cells so that at least a portion of lipid contained within can escape. At least a portion of the lipid can be carried away from the tissue through biological processes or passed from the body by a natural process, e.g., desquamation. In an embodiment where a fat cell is damaged, the fat cell can be viable after treatment. In one embodiment, the treatment radiation can destroy one or more fat cells. In certain embodiments, a first portion of the fat cells are damaged and a second portion are destroyed. In one embodiment, a portion of the fat cells can be removed from the eyelid to selectively change the shape of the eyelid.

In some embodiments, the beam of radiation can be delivered to the target region to thermally injure, damage, and/or destroy one or more fat cells. For example, the beam of radiation can be delivered to a target chromophore in the target region. Suitable target chromophores include, but are not limited to, a fat cell, lipid contained within a fat cell, fatty tissue, a wall of a fat cell, or water in a fat cell or in tissue surrounding a fat cell. The energy absorbed by the chromophore can be transferred to the fat cell to damage or destroy the fat cell. In one embodiment, the beam of radiation is delivered to water and a fat cell in the target region to thermally injure the fat cell.

In various embodiments, treatment radiation can affect one or more fat cells and can cause sufficient thermal injury in the dermal region of the skin to elicit a healing response to cause the skin to remodel itself, resulting in more youthful looking skin. For example, the treatment radiation can partially denature collagen fibers in the target region. Partially denaturing collagen in the dermis can induce and/or accelerate collagen synthesis by fibroblasts. For example, causing selective thermal injury to the dermis can activate fibroblasts, which can deposit increased amounts of extracellular matrix constituents (e.g., collagen and glycosaminoglycans) that can, at least partially, rejuvenate the skin. The thermal injury caused by the radiation can be mild and only sufficient to elicit a healing response and cause the fibroblasts to produce new collagen. Excessive denaturation of collagen in the dermis causes prolonged edema, erythema, and potentially scarring. Inducing collagen formation in the target region can change and/or improve the appearance of the skin of the target region, as well as thicken the skin, tighten the skin, improve skin laxity, and/or reduce discoloration of the skin.

In various embodiments, a treatment can cause minimal cosmetic disturbance so that a patient can return to normal activity following a treatment. For example, a treatment can be performed without causing discernable side effects such as bruising, open wounds, burning, scarring, or swelling. Furthermore, because side effects are minimal, a patient can return to normal activity immediately after a treatment or within a matter of hours, if so desired.

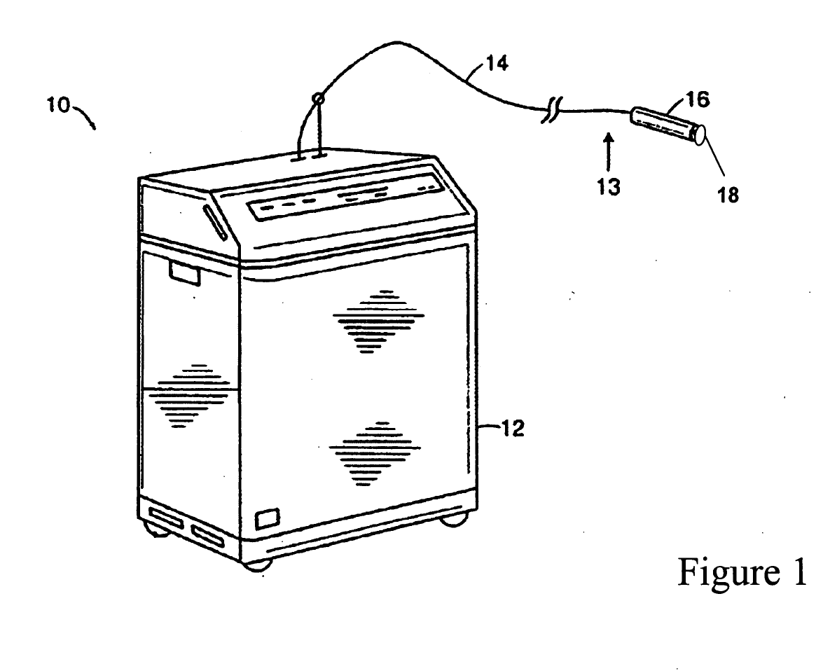

FIG. 1 shows an exemplary embodiment of a system 10 for treating tissue proximate an eye. The system 10 can be used to non-invasively deliver a beam of radiation to a target region. For example, the beam of radiation can be delivered through an external surface of skin over the target region. The system 10 includes an energy source 12 and a delivery system 13. In one embodiment, a beam of radiation provided by the energy source 12 is directed via the delivery system 13 to a target region proximate an eye. In the illustrated embodiment, the delivery system 13 includes a fiber 14 having a circular cross-section and a handpiece 16. A beam of radiation can be delivered by the fiber 14 to the handpiece 16, which can include an optical system (e.g., an optic or system of optics) to direct the beam of radiation to the target region. A user can hold or manipulate the handpiece 16 to irradiate the target region. The delivery system 13 can be positioned in contact with a skin surface, can be positioned adjacent a skin surface, can be positioned proximate a skin surface, can be positioned spaced from a skin surface, or a combination of the aforementioned. In the embodiment shown, the delivery system 13 includes a spacer 18 to space the delivery system 13 from the skin surface. In one embodiment, the spacer 18 can be a distance gauge.

In various embodiments, the energy source 12 can be an incoherent light source (e.g., a broadband source, a flashlamp, and/or an intense pulsed light source), a coherent light source (e.g., a broadband laser or a narrowband laser), a microwave generator, or a radio-frequency generator. In one embodiment, the source generates ultrasonic energy that is used to treat the tissue. In some embodiments, two or more sources can be used together to effect a treatment. For example, an incoherent source can be used to provide a first beam of radiation while a coherent source provides a second beam of radiation. The first and second beams of radiation can share a common wavelength or can have different wavelengths. In an embodiment using an incoherent light source or a coherent light source, the beam of radiation can be a pulsed beam, a scanned beam, or a gated continuous wave (CW) beam. In one embodiment, the source includes an ultrasonic energy device to disrupt or destroy fat cells and a radiation source to induce collagen formation or improve skin laxity.

In various embodiments, the beam of radiation can have one or more wavelengths between about 1000 nm and about 2,600 nm, although longer and shorter wavelengths can be used depending on the application. In some embodiments, the wavelength can be between about 1,000 nm and about 2,200 nm. In other embodiments, the wavelength can be between about 1,160 nm and about 1,800 nm. In yet other embodiments, the wavelength can be between about 1,300 nm and about 1,600 nm. In one embodiment, the wavelength is about 1,200 nm or about 1,750 nm. In one detailed embodiment, the wavelength is about 1,208 nm, 1,270 nm, 1,310 nm, 1,450 nm, 1,550 nm, 1,720 nm, 1,930 nm, or 2,100 nm. One or more of the wavelengths used can be within a range of wavelengths that is transmitted to the tissue proximate the eye. Furthermore, the beam of radiation can be a single wavelength device or include a band of wavelengths.

In an embodiment having a second beam of radiation targeting blood, blood vessels, and/or a component of blood to reduce the discoloration, the wavelength can be between about 400 nm and about 1,300 nm, although longer and shorter wavelengths can be used depending on the application. In some embodiments, the second beam of radiation has a wavelength between about 400 nm and about 800 nm. In one embodiment, the second beam of radiation has a wavelength of 532 nm, 585 nm, 595 nm, 694 nm, 755 nm, or 1064 nm. In some embodiments, the second beam of radiation is provided by an incoherent, broadband radiation source.

In various embodiments, the beam of radiation can have a fluence between about 5 J/cm² and about 100 J/cm², although higher and lower fluences can be used depending on the application. In some embodiments, the fluence can be between about 10 J/cm² and about 100 J/cm².

In various embodiments, the beam of radiation can have a spotsize between about 0.5 mm and about 25 mm, although larger and smaller spotsizes can be used depending on the application.

In various embodiments, the beam of radiation can have a pulsewidth between about 10 μs and about 30 s, although larger and smaller pulsewidths can be used depending on the application.

In various embodiments, the beam of radiation can be delivered at a rate of between about 0.1 pulse per second and about 10 pulses per second, although faster and slower pulse rates can be used depending on the application.

In various embodiments, the parameters of the radiation can be selected to deliver the beam of radiation to a predetermined depth. In some embodiments, the beam of radiation can be delivered to the target region about 0.1 mm to about 3 mm below an exposed surface of the skin, although shallower or deeper depths can be selected depending on the application.

In various embodiments, the tissue can be heated to a temperature of between about 50° C. and about 80° C., although higher and lower temperatures can be used depending on the application. In one embodiment, the temperature is between about 55° C. and about 70° C.

To minimize thermal injury to tissue surrounding an eye and/or to an exposed surface of the target region, the delivery system 13 shown in FIG. 1 can include a cooling system for cooling before, during or after delivery of radiation. Cooling can include contact conduction cooling, evaporative spray cooling, convective air flow cooling, or a combination of the aforementioned. In one embodiment, the handpiece 16 includes a skin contacting portion that can be brought into contact with the skin. The skin contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the skin.

A spray cooling device can use cryogen, water, or air as a coolant. In one embodiment, a dynamic cooling device can be used to cool the skin (e.g., a DCD available from Candela Corporation). For example, the delivery system 13 shown in FIG. 1 can include tubing for delivering a cooling fluid to the handpiece 16. The tubing can be connected to a container of a low boiling point fluid, and the handpiece can include a valve for delivering a spurt of the fluid to the skin. Heat can be extracted from the skin by virtue of evaporative cooling of the low boiling point fluid. The fluid can be a non-toxic substance with high vapor pressure at normal body temperature, such as a Freon or tetrafluoroethane.

In various embodiments, a delivery system can include a focusing system for focusing the beam of radiation below the surface of the skin in the target region to affect at least one fat cell. The focusing system can direct the beam of radiation to the target region about 0.1 mm to about 3 mm below the exposed surface of the skin. In some embodiments, the delivery system can include a lens, a planoconvex lens, or a plurality of lens to focus the beam of radiation.

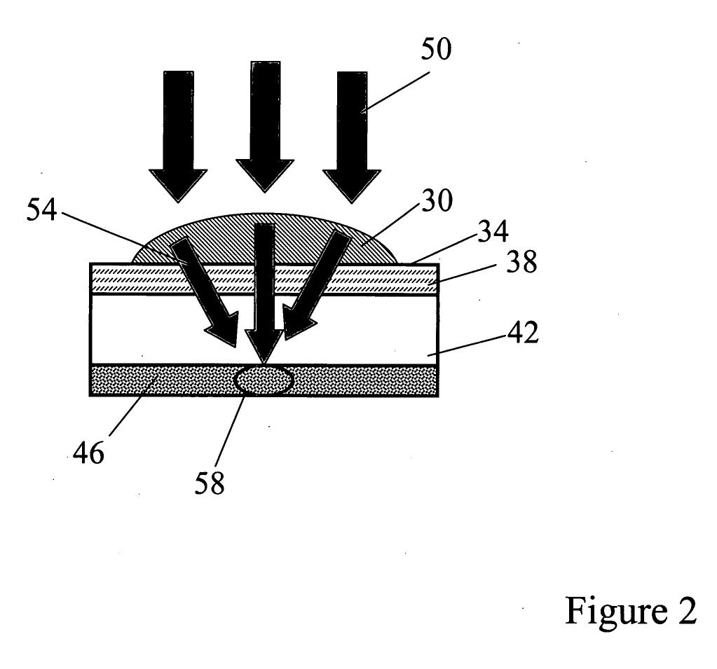

FIG. 2 shows a planoconvex lens 30 positioned on a surface 34 of a section of skin, including an epidermal region 38, a dermal region 42, and a layer of fatty tissue 46. The planoconvex lens 30 focuses radiation 50 (focusing shown by arrows 54) to a sub surface focal region 58, which can include at least one fat cell.

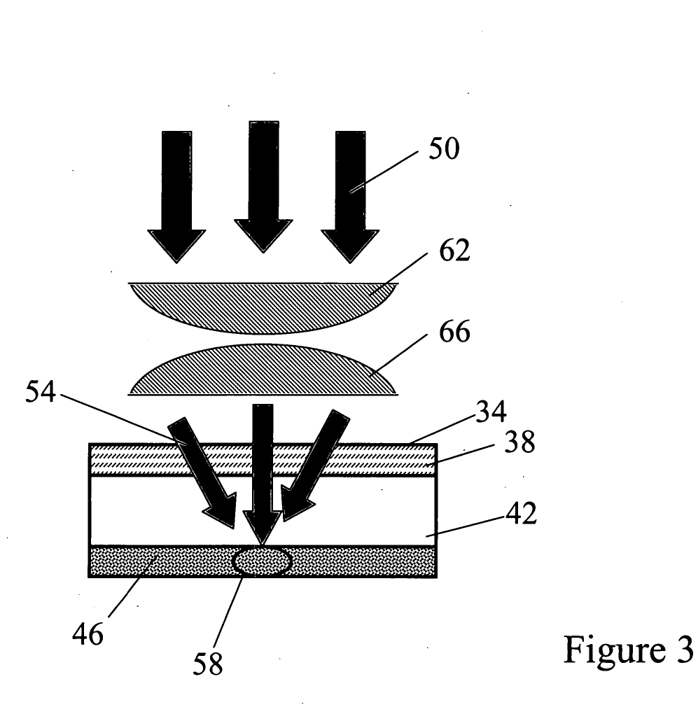

FIG. 3 shows a plurality of lens 62, 66 spaced from the skin surface 34. The plurality of lens 62, 66 focus the radiation 50 (focusing shown by the arrows 54) to the sub surface focal region 58.

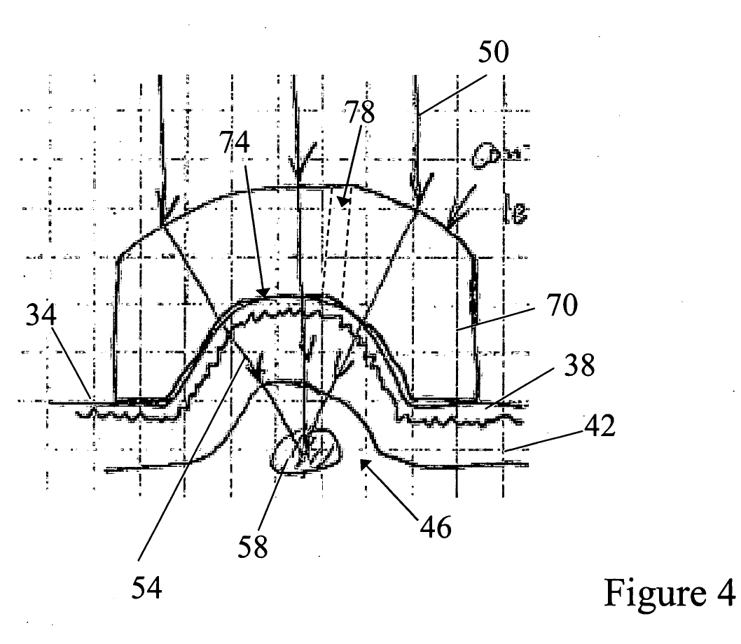

FIG. 4 shows a lens 70 having a concave surface 74 for contacting the skin surface 34. In certain embodiments, the lens 70 is placed proximate to a target region of skin. Vacuum can be applied to draw the target region of skin against the concave surface 74 of the lens 70. Vacuum can be applied through orifice 78 in the lens 70 by a vacuum device. The lens 70 focuses the radiation 50 to the sub surface focal region 58.

In various embodiments, the source of radiation can be a diode laser having sufficient power to affect one or more fat cells. An advantage of diode lasers is that they can be fabricated at specific wavelengths that target fatty tissue. A limitation, though, of many diode laser devices and solid state devices targeting fatty tissue is the inability to produce sufficient power to effectuate a successful treatment.

In one embodiment, a diode laser of the invention is a high powered semiconductor laser. In one embodiment, the source of radiation is a fiber coupled diode laser array. For example, an optical source of radiation can include a plurality of light sources (e.g., semiconductor laser diodes) each adapted to emit a beam of light from a surface thereof. A plurality of first optical fibers each can have one end thereof adjacent the light emitting surface of a separate one of the light sources so as to receive the beam of light emitted therefrom. The other ends of the first optical fibers can be bundled together in closely spaced relation so as to effectively emit a single beam of light, which is a combination of the beams from all of the first optical fibers. A second optical fiber can have an end adjacent the other ends of the first optical fibers to receive the beam of light emitted from the bundle of first optical fibers. The beam of light from the bundled other ends of the first optical fibers can be directed into the second optical fiber. The first optical fiber can have a numerical aperture less than that of the second fiber. An exemplary fiber coupled diode laser array is described in U.S. Pat. No. 5,394,492, owned by the assignee of the instant application and the entire disclosure of which is herein incorporated by reference.

While the invention has been particularly shown and described with reference to specific illustrative embodiments, it should be understood that various changes in form and detail may be made without departing from the spirit and scope of the invention.

What is claimed:

1. A method of altering the appearance of a sagging eyelid, comprising:
cooling a surface of a target region of skin causing the sagging eyelid; and
delivering a beam of radiation to the target region to affect at least one fat cell of a fatty deposit to alter the appearance of the sagging eyelid without causing substantial unwanted injury to tissue surrounding the target region.

2. The method of claim 1 further comprising causing the fatty deposit of the eyelid to be reduced in size.

3. The method of claim 1 further comprising damaging the at least one fat cell so that lipid contained within can escape and at least a portion of the lipid can be carried away from the tissue.

4. The method of claim 1 further comprising destroying the at least one fat cell.

5. The method of claim 1 further comprising delivering the beam of radiation to the target region to thermally injure the at least one fat cell.

6. The method of claim 1 further comprising reducing discoloration of skin of the target region.

7. The method of claim 1 further comprising inducing collagen formation in the target region of skin to improve skin laxity.

8. The method of claim 1 further comprising delivering the beam of radiation to the target region about 0.1 mm to about 3 mm below the exposed surface of the skin.

9. The method of claim 1 further comprising focusing the beam of radiation below the surface of the skin in the target region to affect the at least one fat cell.

10. The method of claim 9 further comprising focusing the beam of radiation using a planoconvex lens.

11. The method of claim 9 further comprising:
providing a lens with a concave contact surface; and
applying vacuum to draw the target region of skin against the concave contact surface of the lens to focus the beam of radiation to the at least one fat cell in the target region.

12. An apparatus for altering the appearance of a sagging eyelid, comprising:
a source generating a beam of radiation, the source including a fiber coupled laser diode array;
a delivery system coupled to the source for directing the beam of radiation to a target region of skin to affect at least one fat cell of a fatty deposit to alter the appearance of the sagging eyelid; and
a cooling system for cooling a surface of the target region of skin to minimize unwanted injury to tissue surrounding the target region.

13. The apparatus of claim 12 wherein the beam of radiation improves the appearance of the fatty deposit of the eyelid.

14. The apparatus of claim 12 wherein the beam of radiation causes the fatty deposit of the eyelid to be reduced in size.

15. The apparatus of claim 12 wherein the delivery system directs the beam of radiation to the target region about 0.1 mm to about 3 mm below the surface of the skin.

16. The apparatus of claim 12 wherein the fiber coupled laser diode array generates a beam of radiation having at least one wavelength of about 1,208 nm, 1,270 nm, 1,310 nm, 1,450 nm, 1,550 nm, 1,720 nm, 1750 nm, 1,930 nm, and 2,100 nm.

17. The apparatus of claim 12 further comprising a focusing system to focus the beam of radiation below the surface of the skin to the fatty deposit in the target region to affect the at least one fat cell.

18. The apparatus of claim 17 wherein the focusing system comprises a planoconvex lens to focus the beam of radiation.

19. The apparatus of claim 17 wherein the focusing system comprises a plurality of lens to focus the beam of radiation.

20. The apparatus of claim 17 further comprising a vacuum system and wherein the focusing system comprises a lens with a concave surface to contact the surface of the skin, vacuum being applied to draw the target region of skin against the concave surface of the lens so that the lens focuses the beam of radiation to the at least one fat cell in the target region.

21. The apparatus of claim 17 wherein the focusing system directs the beam of radiation to the target region about 0.1 mm to about 3 mm below the surface of the skin.

22. The apparatus of claim 12 wherein the fiber coupled laser diode array comprises a high power semiconductor laser.

23. An apparatus for altering the appearance of a sagging eyelid, comprising:
means for cooling a surface of a target region of skin causing the sagging eyelid; and
means for delivering a beam of radiation to the target region to affect at least one fat cell of a fatty deposit to alter the appearance of the sagging eyelid without causing substantial unwanted injury to tissue surrounding the target region.
